(19) United States
(12) Patent Application Publication
(10) Pub. No.: US 2015/0017672 A1
(43) Pub. Date: Jan. 15, 2015

(54) ASSAYS FOR DETECTION OF GLYCOSAMINOGLYCANS

(71) Applicant: SHIRE HUMAN GENETIC THERAPIES INC., Lexington, MA (US)

(72) Inventors: Juan Ruiz, Acton, MA (US); Marcia Sellos-Moura, West Newbury, MA (US); Philip Shi, Newton, MA (US)

(21) Appl. No.: 14/375,823

(22) PCT Filed: Feb. 1, 2013
(86) PCT No.: PCT/US2013/024379

§ 371 Date: Jul. 31, 2014

Related U.S. Application Data
(60) Provisional application No. 61/593,777, filed on Feb. 1, 2012.

(51) Int. Cl. C12Q 1/37

(52) U.S. Cl. CPC C12Q 1/37; G01N 2800/04; G01N 2500/20
USPC 435/23

(57) ABSTRACT

Disclosed herein are novel methods, assays and kits useful for the diagnosis and monitoring of subjects with mucopolysaccharidoses (MPS). The methods, assays and kits are particularly useful for detecting the presence of one or more glycosaminoglycans which correlate to MPS and its severity in a variety of biological samples.

### RELATED APPLICATION

The subject application claims priority to, and the benefit of, U.S. Provisional Application No. 61/593,777, filed Feb. 1, 2012, the entire teachings of which are incorporated herein by reference.

### BACKGROUND OF THE INVENTION

The mucopolysaccharidoses (MPS) represent a group of rare, inherited lysosomal storage disorders caused by the deficiency or inactivity of lysosomal enzymes. In particular, the MPS disorders are caused by the deficiency or inactivity of the lysosomal enzymes which catalyze the stepwise metabolism of complex sugar molecules known as glycosaminoglycans (GAGs). These enzyme deficiencies in turn result in the accumulation of GAGs in the cells, tissues, and, in particular, cellular lysosomes of affected subjects, resulting in permanent, progressive cellular damage which affects appearance, physical abilities, organ and system functioning, and, in most cases, mental development of affected subjects.

Eleven discrete enzyme deficiencies have been identified, which result in seven distinct clinical classes of MPS. Each MPS disorder is characterized by a deficiency or inactivity of an enzyme involved in the metabolism of one or more of the GAGs heparan sulfate (HS), dermatan sulfate (DS), chondroitin sulfate (CS), keratan sulfate (KS) or hyaluronan. As a result, such GAGs accumulate in the cells and tissues of affected subjects.

The diagnosis of MPS and the monitoring of both disease progression and treatment efficacy rely on subjective and objective monitoring of the subject. Available objective assays useful for the diagnosis of MPS and for monitoring treatment efficacy have traditionally lacked sensitivity, have significant limitations, and have been characterized as inadequate (see for example, Oguma T, et al., Analytical Biochemistry (2007) 368: 79-86.)

Diagnosis of MPS can be made through urinalysis. For example, the use of diagnostic methods which rely on spectrophotometry to measure total GAGs in urine are based upon binding to dimethylmethylene blue, and are limited to use in urinary assays. Furthermore, the utility of urinalysis determinations, may not be indicative of a specific MPS disorder because the presence of excess GAGs in the urine of a subject may only provide objective evidence that either one of the several MPS disorders are present. (Neufeld E F, et al., The mucopolysaccharidoses. In: Scriver C R, ed. The Metabolic and Molecular Bases of Inherited Disease. New York, N.Y.: McGraw-Hill (2001) 3421-3452.) Similarly, urinalysis assays which analyze GAGs content in the urine are not particularly sensitive and a negative urine GAGs test may not necessarily preclude a diagnosis of MPS. Reported assays based on the use of enzymatic digestion of GAGs and high-performance liquid chromatography (HPLC) detection of the corresponding disaccharides are also limited, for example due to their lack of specificity and/or sensitivity.

Reported assays which measure biological markers correlating to MPS also have limitations. For example, assays which measure serum levels of a heparin cofactor-II-thrombin complex (THC) have been described as a monitoring tool for subjects with MPS. (Randall, D R, et al. Molecular Genetics and Metabolism (2008), 94: 456-461.) Such assays however, are of limited use in MPS classes where the accumulating pathological GAG is not primarily dermatan sulfate, such as in MPS-III, MPS-IV, MPS-VII or MPS-IX, since under physiological conditions formation of THC is not efficiently catalyzed by HS, CS, KS or hyaluronan.

New screening methods, assays and biological markers are needed to diagnosis MPS and to monitor clinical course and disease progression/regression before, during and after treatment. In particular new assays that are useful for quantifying GAGs in a variety of biological samples (e.g., serum, urine, and CSF) would be useful to monitor disease severity, progression and treatment efficacy.

### SUMMARY OF THE INVENTION

The present invention relates to novel methods, assays and kits useful for identifying subjects having mucopolysaccharidoses (MPS), as well as monitoring disease severity, progression and treatment efficacy. The methods, assays and kits of the present invention are particularly useful for detecting the presence or absence of one or more glycosaminoglycans (e.g., dermatan sulfate and heparan sulfate) in a variety of biological samples (e.g., serum, urine, and CSF) and provide a means of diagnosing MPS, determining MPS severity, or alternatively for determining the responsiveness of MPS to therapeutic agents and regimens administered to a subject for the treatment of MPS.

As described herein, assays are disclosed which combine a serine protease, an inhibitor of the serine protease, and a substrate for the serine protease, along with one or more glycosaminoglycans (e.g., in a sample), under conditions suitable for cleavage of the serine protease substrate by the serine protease. Presence of the serine protease inhibitor inhibits the activity of the serine protease on its substrate, and presence of the one or more glycosaminoglycans improves, catalyzes or facilitates this inhibition. As a result, cleavage of the serine protease substrate is inhibited to a greater extent in the presence of one or more glycosaminoglycans than in the absence of glycosaminoglycans. Substrates suitable for use in the assays will typically be chromogenic or fluorogenic substrates whose cleavage products are detectable by spectrophotometric means. After the components of the assay are combined for a suitable time period, spectrophotometric analysis is performed, and the results are compared against a standard which calibrates absorbance with glycosaminoglycan concentration. In this way the concentration of glycosaminoglycan in the sample can be determined.

In certain embodiments, the methods and assays of the present invention may comprise a step of determining the quantity and/or concentration of one or more glycosaminoglycans in a biological sample by comparatively assessing the concentration of such glycosaminoglycans (e.g., using spectroscopy) relative to one or more standard or calibration curves which have been constructed using standard solutions with known quantities of such glycosaminoglycans. For example, the concentrations of the glycosaminoglycan heparan sulfate (HS) in one or more biological samples may be determined with reference to one or more calibration curves generated to correlate spectrophotometric absorbance with glycosaminoglycan concentration (e.g., calibration curves that are prepared using HS calibrator samples serially diluted from about 20 ng/mL to about 0.6 ng/mL in a selected assay buffer solution and that are sequentially incubated with heparin cofactor II, human thrombin (at a final concentration of 0.1-0.2 μg/mL) and a suitable thrombin substrate). Alternatively, the methods and assays of the present invention may be used to determine the concentration of one or more GAGs by comparison to one or more controls.

Also contemplated are kits for determining the concentration and/or quantity of one or more glycosaminoglycans. Such kits preferably include one or more reagents (e.g., one or more of a serine protease, a labeled substrate for said serine protease, an inhibitor of said serine protease, standards, buffers, instructions for carrying out a method in accordance with the present invention, and combinations thereof).

In certain embodiments, the present inventions are directed to one or more assay buffer solutions (e.g., an assay buffer solution comprising HEPES, NaCl and PEG). Such assay buffer solutions may provide a means of optimizing the sensitivity of the assays disclosed herein. In certain embodiments, the concentration of sodium chloride (NaCl) in the assay buffer may provide a means of controlling, modulating or otherwise improving the sensitivity of the assay (e.g., the thrombin-coupled assays). For example, the sensitivity of the thrombin-coupled assays disclosed herein to detect one or more GAGs (e.g., dermatan sulfate (DS), heparan sulfate (HS), chondroitin sulfate, keratan sulfate or hyaluronan) in a sample may be controlled, and in certain instances optimized, based on the concentration of sodium chloride. For instance, an optimized assay buffer solution for detecting the concentration of dermatan sulfate (DS) in a sample may comprise about 40-60 mM NaCl (e.g., about 50 mM NaCl). Similarly, an optimized assay buffer solution for detecting the concentration of heparan sulfate (HS) in a sample may comprise approximately 40-60 mM NaCl (e.g., about 40 mM NaCl or about 50 mM NaCl) or approximately 30-60 mM NaCl (e.g., about 30 mM NaCl).

Disclosed is a method for determining the concentration of one or more glycosaminoglycans in a sample comprising (a) combining a serine protease (e.g., of the clotting cascade), a labeled substrate for said serine protease, an inhibitor of said serine protease, and a sample suspected of comprising one or more glycosaminoglycans under conditions and for a time suitable for cleavage of the labeled substrate by the serine protease to produce a detectable signal, (b) detecting the detectable signal, and (c) comparing the amount of detectable signal with a standard to determine the concentration of said one or more glycosaminoglycans in said sample, wherein said inhibitor of said serine protease is selected from the group consisting of heparin cofactor II and antithrombin III, and wherein said one or more glycosaminoglycans are selected from the group consisting of dermatan sulfate (DS) and heparan sulfate (HS).

Also disclosed is a method of identifying an individual having a mucopolysaccharidosis (MPS), comprising determining the concentration of one or more glycosaminoglycans in a biological sample obtained from said individual by a method comprising (a) combining a serine protease (e.g., of the clotting cascade), a labeled substrate for said serine protease, an inhibitor of said serine protease, and a biological sample from said subject under conditions and for a time suitable for cleavage of the labeled substrate by the serine protease to produce a detectable signal, (b) detecting the detectable signal, and (c) comparing the amount of detectable signal with a standard to determine the concentration of said one or more glycosaminoglycans in said sample, wherein said inhibitor of said serine protease is selected from the group consisting of heparin cofactor II and antithrombin III, wherein said one or more glycosaminoglycans are selected from the group consisting of dermatan sulfate (DS) and heparan sulfate (HS), and wherein the concentration of said one or more glycosaminoglycans is indicative of whether the individual has MPS.

Also disclosed is a method of determining the efficacy of one or more therapeutic agents or regimens for treatment of mucopolysaccharidosis (MPS) comprising determining the concentration of one or more glycosaminoglycans in a first biological sample obtained from an individual prior to administration of one or more therapeutic agents or regimens to said individual, determining the concentration of said glycosaminoglycans in a second biological sample obtained from said individual after administration of one or more therapeutic agents or regimens to said individual, wherein if the concentration of said glycosaminoglycans in said second biological sample is lower than the concentration in said first biological sample the one or more therapeutic agents or regimens are efficacious for treatment of MPS, and wherein determining the concentration of one or more glycosaminoglycans in the first and second biological samples is performed by a method comprising (a) combining a serine protease (e.g., of the clotting cascade), a labeled substrate for said serine protease, an inhibitor of said serine protease, and the first or second biological sample under conditions and for a time suitable for cleavage of the labeled substrate by the serine protease to produce a detectable signal, (h) detecting the detectable signal, and (c) comparing the amount of detectable signal with a standard to determine the concentration of said one or more glycosaminoglycans in said sample, wherein said inhibitor of said serine protease is selected from the group consisting of heparin cofactor II and antithrombin III, and wherein said one or more glycosaminoglycans are selected from the group consisting of dermatan sulfate (DS) and heparan sulfate (HS).

Also disclosed is a method of determining the progression of a mucopolysaccharidosis (MPS) disorder in an individual, comprising determining the concentration of one or more glycosaminoglycans in a first biological sample obtained from an individual and determining the concentration of said one or more glycosaminoglycans in one or more subsequent biological samples obtained from said individual, wherein if the concentration of said one or more glycosaminoglycans in said one or more subsequent biological samples is greater than the concentration in said first biological sample it is indicative that the MPS is progressing, and wherein determining the concentration of one or more glycosaminoglycans in the first and subsequent biological samples is performed by a method comprising (a) combining a serine protease, a labeled substrate for said serine protease, an inhibitor of said serine protease, and the first or subsequent biological sample under conditions and for a time suitable for cleavage of the labeled substrate by the serine protease to produce a detectable signal, (b) detecting the detectable signal, and (c) comparing the amount of detectable signal with a standard to determine the concentration of said one or more glycosaminoglycans in said sample, wherein the serine protease is a serine protease of the clotting cascade, wherein said inhibitor of said serine protease is selected from the group consisting of heparin cofactor II and antithrombin III, and wherein said one or more glycosaminoglycans are selected from the group consisting of dermatan sulfate (DS) and heparan sulfate (HS).

In some embodiments the serine protease is selected from the group consisting of the serine proteases shown in FIG. 7 and FIG. 8.

In some embodiments the labeled substrate is a chromogenic or fluorogenic substrate.

In some embodiments the serine protease is thrombin and the labeled substrate is a chromogenic thrombin substrate.

In some embodiments the sample is treated to inactivate all but one of the glycosaminoglycans in the sample. In some embodiments the sample is treated with chondroitinase B and the active glycosaminoglycan is dermatan sulfate. In some embodiments the sample is treated with heparinases and the active glycosaminoglycan is heparan sulfate.

In some embodiments detecting the detectable signal is performed by spectrophotometric detection. In some embodiments the spectrophotometric detection is performed at 405 nm.

In some aspects the sample is a biological sample. In some aspects the sample is selected from the group consisting of urine, serum, cerebrospinal fluid, and saliva.

In some embodiments the standard is a curve which calibrates spectrophotometric absorbance with glycosaminoglycan concentration.

In some aspects the MPS is selected from the group consisting of MPS I, MPS II, MPS IIIA, MPS IIIB, MPS IIIC, MPS IIID, MPS IVA, MPS IVB, MPS VI, MPS VII and MPS IX.

In some aspects the one or more therapeutic agents or regimens are selected from the group consisting of enzyme replacement therapies, bone marrow transplantation, and combinations thereof. In some embodiments the one or more therapeutic agents are selected from the group consisting of iduronate sulfatase, idursulfase, alpha-L-iduronidase, heparin sulfamidase, N-acetylglucosaminidase, N-acetylglucosamine 6-sulfatase, N-acetylgalactosamine-4-sulfatase and beta-glucoronidase.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 illustrates sample Dermatan Sulfate (DS) Standard Curve in Assay Buffer 1.

FIG. 2 illustrates sample Dermatan Sulfate (DS) Calibrator absorbance values in Assay Buffer 1.

FIG. 3 illustrates sample Heparan Sulfate (HS) Standard Curve in Assay Buffer 2

FIG. 4 illustrates sample Heparan Sulfate (HS) Calibrator absorbance values in Assay Buffer 2.

FIG. 5 illustrates sample Heparan Sulfate (HS) Standard Curve in 5% cerebrospinal fluid (CSF) in Assay Buffer 2.

FIG. 6 illustrates sample Heparan Sulfate (HS) Calibrator absorbance values in 5% cerebrospinal fluid (CSF) in Assay Buffer 2.

FIG. 7 illustrates the various enzymes and enzymatic actions involved in the proteolytic cascade of the coagulation pathway and shows serine proteases of the clotting (or complement) cascade.

FIG. 8 illustrates how the generation of thrombin can be divided into three phases, the intrinsic and extrinsic pathways, which provide alternative routes for the generation of factor X, and the final common pathway, which results in thrombin formation.

FIG. 9 illustrates the results of an ongoing observational natural history study conducted using the thrombin-coupled assays described herein. Subjects having MPS-IIIA and healthy control subjects were evaluated over the course of a one year period. A cerebrospinal fluid (CSF) sample was obtained from each subject by lumbar puncture upon enrollment and again at six and twelve months. The concentration of HS in the CSF samples was determined using the thrombin-coupled assays of the present invention. As illustrated in FIG. 9, concentrations of HS in the CSF samples obtained from subjects having MPS-IIIA are elevated and appear to remain relatively constant for up to 12 months. The results presented in FIG. 9 therefore demonstrate the ability of the thrombin-coupled assays disclosed herein to measure GAG concentration in a biological sample and further illustrate that GAG concentrations in a biological sample (e.g., CSF) may be used as an objective indicator to characterize disease severity and to monitor response to therapeutic intervention.

FIG. 10 comparatively illustrates the effects of sodium chloride concentration of assay buffer solutions on the sensitivity of thrombin-coupled assays used to detect dermatan sulfate. As shown in FIG. 10, a lower percentage of thrombin inhibition was observed using an assay buffer solution that comprised 100 mM of sodium chloride, whereas a flatter inhibition curve was observed using an assay buffer solution that excluded sodium chloride (0.0 mM). In contrast, the assay buffer solutions comprising between 40 mM and 60 mM NaCl produced sensitive and steep calibration curves. Based on these findings, it was determined that to achieve an optimum sensitivity of the thrombin-coupled assays to detect dermatan sulfate, the assay buffer solution should have between about 40-60 mM NaCl. Optimized assay buffer solutions comprising between about 40 mM NaCl and 60 mM NaCl therefore demonstrate optimum sensitivity to detect dermatan sulfate using the thrombin-coupled assays disclosed herein.

FIG. 11 comparatively illustrates the effects of sodium chloride concentration of assay buffer solutions on the sensitivity of thrombin-coupled assays used to detect heparan sulfate. As depicted in FIG. 11, a lower percentage of thrombin inhibition was observed using an assay buffer solution that comprised 75 mM of sodium chloride, whereas a flatter inhibition curve was observed using assay buffer solutions that comprised 10 mM or 25 mM of sodium chloride. In contrast, the assay buffer solution comprising 50 mM NaCl produced a sensitive and steep thrombin inhibition curve. Based on these findings, it was determined that to achieve an optimum sensitivity of the thrombin-coupled assays to detect heparan sulfate, the assay buffer solution should have about 40-50 mM NaCl (e.g., 40 mM NaCl). Optimized assay buffer solutions comprising about 40-50 mM NaCl (e.g., 40 mM NaCl) therefore demonstrate optimum sensitivity to detect heparan sulfate using the thrombin-coupled assays disclosed herein.

### DETAILED DESCRIPTION OF THE INVENTION

The present invention is based on the discovery that under optimized conditions glycosaminoglycans (e.g., dermatan sulfate and heparan sulfate) may be indirectly measured in a biological sample, and provide a means of diagnosing MPS and/or monitoring disease progression or the efficacy of a therapeutic agent and/or regimen administered to a subject with MPS. Specifically, the present invention relates to novel methods, assays and kits useful for identifying subjects having MPS and for measuring or monitoring response to therapy and disease progression. The methods, assays and kits of the present invention are particularly useful for indirectly detecting the presence of and/or quantifying one or more glycosaminoglycans in a variety of biological samples (e.g., serum, urine, and CSF) and thus provide a means of diagnosing MPS, determining MPS severity, or alternatively for determining the responsiveness of MPS to therapeutic agents administered for the treatment of MPS. The MPS disorders are lysosomal storage diseases, characterized by malfunctioning lysosomes. As used herein, the terms “mucopolysaccharidoses” or “MPS” mean any of a group of genetic disorders involving a defect in the metabolism of glycosaminoglycans resulting in greater than normal levels of such glycosaminoglycans in the cells and tissues of a subject. Examples of MPS disorders and the corresponding deficient enzymes and accumulated GAGs are shown below in Table 1.

TABLE 1
MPS Disorders

| | | | ACCUMULATED |
|---|---|---|---|
| TYPE | DISEASE | DEFICIENT ENZYME | GAGs |
| MPS-I | Hurler syndrome | α-L-iduronidase | HS, DS |
| MPS-II | Hunter syndrome | Iduronate 2-sulfatase | HS, DS |
| MPS-IIIA | Sanfilippo syndrome A | Heparan sulfate N-sulfatase | HS |
| MPS-IIIB | Sanfilippo syndrome B | N-acetyl-α-D-glucosaminidase | HS |
| MPS-IIIC | Sanfilippo syndrome C | acetyl-CoA-α-glucosaminide | HS |
| | | N-acetyltransferase | |
| MPS-IIID | Sanfilippo syndrome D | N-acetylgucosamine-6- | HS |
| | | sulfatase | |
| MPS-IVA | Morquio syndrome A | N-acetylgalactosamine-6- | KS, CS |
| | | sulfatase | |
| MPS-IVB | Morquio syndrome B | β-galactosidase | KS |
| MPS-VI | Maroteaux-Lamy | N-acetylgalactosamine-4- | DS |
| | syndrome | sulfatase | |
| MPS-VII | Sly syndrome | β-glucuronidase | HS, DS, CS |
| MPS-IX | Natowicz syndrome | Hyaluronidase | Hyaluronic acid |

Subjects with MPS either do not produce enough of one of the 11 enzymes required to metabolize GAGs into degradation products and simpler disaccharide molecules, or produce enzymes that do not function properly. Over time, the GAGs accumulate in the cells, blood, and connective tissues, resulting in permanent, progressive cellular damage that affects the subject's appearance, physical abilities, organ and system functioning, and, frequently, mental development.

There is currently no cure for MPS, and treatment options are limited. Palliative care may be offered to subjects to improve their quality of life. Additionally, bone marrow transplantation may be considered a viable therapeutic option for some subjects with MPS, although the availability and efficacy of bone marrow transplant is frequently limited to subjects who are strong enough to endure the procedure.

Enzyme replacement therapy (ERT) has been shown to be a useful therapeutic alternative for some MPS subjects. For example, clinical studies have demonstrated that administration of recombinant alpha-L-iduronidase can alter the phenotype of MPS I patients to varying degrees (Wraith, J E, et al., J. Pediatr. (2004) 144: 581-588). The phrase “therapeutic agent” as used herein, refers to any treatment anticipated to affect (i.e., improve or slow progression) MPS disease progression (e.g., as objectively measured using the methods, assays and kits described herein), and include, without limitation ERT (e.g., alpha-L-iduronidase, iduronate 2-sulfatase (such as idursulfase), heparin sulfamidase, N-acetylglucosaminidase, N-acetylglucosamine 6-sulfatase, N-acetylgalactosamine-4-sulfatase and beta-glucoronidasc) and bone marrow transplants. In accordance with one embodiment of the present invention, the kits, assays and methods of the present invention may be used to monitor disease progression or the efficacy of a therapeutic agent administered to a subject.

The methods, assays and kits of the present invention may advantageously be employed using various biological samples. As the phrase is used herein, “biological sample” means any sample taken or derived from a subject. Examples of contemplated biological samples include cerebrospinal fluid, tissues such as chorionic villus, cell samples, organs, biopsies, blood, serum, amniotic fluid, saliva and urine. In some embodiments the biological sample will contain additional proteins. Also contemplated by the present invention is a differential analysis of more than one biological sample obtained from a subject (e.g., two, three, four, five, six or more biological samples), for example to monitor disease progression, regression or treatment efficacy. Such samples may be distinguished herein by reference to a first biological sample and a second (or subsequent) biological sample. In a preferred embodiment, such first biological sample and such second biological samples are obtained from a subject at varying intervals (e.g., before and after the administration of a therapeutic agent or initiation of MPS therapy). Similarly, biological samples may be at multiple time points, e.g., collected over the course of a subject's lifetime, to monitor disease progression, and comparative analyses of such samples may be used to inform treatment decisions (e.g., increasing or decreasing the dose of a therapeutic agent, or discontinuing a therapeutic agent in favor of initiating another therapeutic agent or regimen). The skilled artisan will be able to make MPS diagnostic, prognostic, and progressive determinations based on art-recognized correlations between GAG concentrations/amounts and MPS status, optionally in combination with other diagnostic and prognostic indicators.

The methods, assays and kits of the present invention advantageously provide a means of measuring MPS disease progression, e.g., in the central nervous system (CNS), by assaying cerebrospinal fluid (CSF). The ability to assay CSF is particularly useful for the monitoring of subjects with MPS disorders that are characterized as having a CNS etiology, such as Sanfilippo syndrome and Hunter syndrome.

In the context of the present invention, the term “subject” refers to a mammal, and the term “individual” refers to a human. Contemplated subjects and individuals include those suspected of having an MPS disorder and those with a confirmed MPS disorder.

The methods and assays of the present invention are directed to measuring the accumulated glycosaminoglycans as a means of either diagnosing MPS or monitoring disease progression or treatment efficacy. The terms “glycosaminoglycan” or “GAGs” refer to sulfated heteropolysaccharide molecules of varying lengths containing repeating disaccharide units. In some embodiments of the present invention, the GAGs are exemplified by heparan sulfate (HS), dermatan sulfate (DS), chondroitin sulfate, keratan sulfate or hyaluronan. In certain embodiments, the GAG is not heparin, for example, as indicated by pre-treatment of the samples with a heparinase (e.g., herarinase I, II and/or III).

Specifically, the assays and methods of the present invention rely on one or more glycosaminoglycans as a biological marker or an indicator of a biologic state (e.g., MPS) and may include a characteristic that is objectively measured as an indicator of normal biological processes, pathologic processes, or pharmacologic responses to a therapeutic or other intervention. In one particular embodiment of the present invention, the glycosaminoglycans are quantified indirectly. For example, the presence or quantity of a glycosaminoglycan may be assessed by analyzing such glycosaminoglycan's ability to catalyze a known enzymatic reaction. In one embodiment of the present invention, the glycosaminoglycans may be indicative of the presence of MPS or alternatively may be indicative of a MPS progression or regression (e.g., in response to the administration of a therapeutic agent.) In a preferred embodiment, the GAGs of the present invention comprise heparan sulfate (HS), dermatan sulfate (DS), chondroitin sulfate (CS), keratan sulfate (KS), hyaluronan and combinations thereof.

The methods and assays of the present invention are particularly sensitive and surprisingly retain such sensitivity irrespective of the selected biological sample. For example, the methods and assays of the present invention are capable of detecting low (e.g., on a nanogram scale) concentrations or quantities of one or more GAGS.

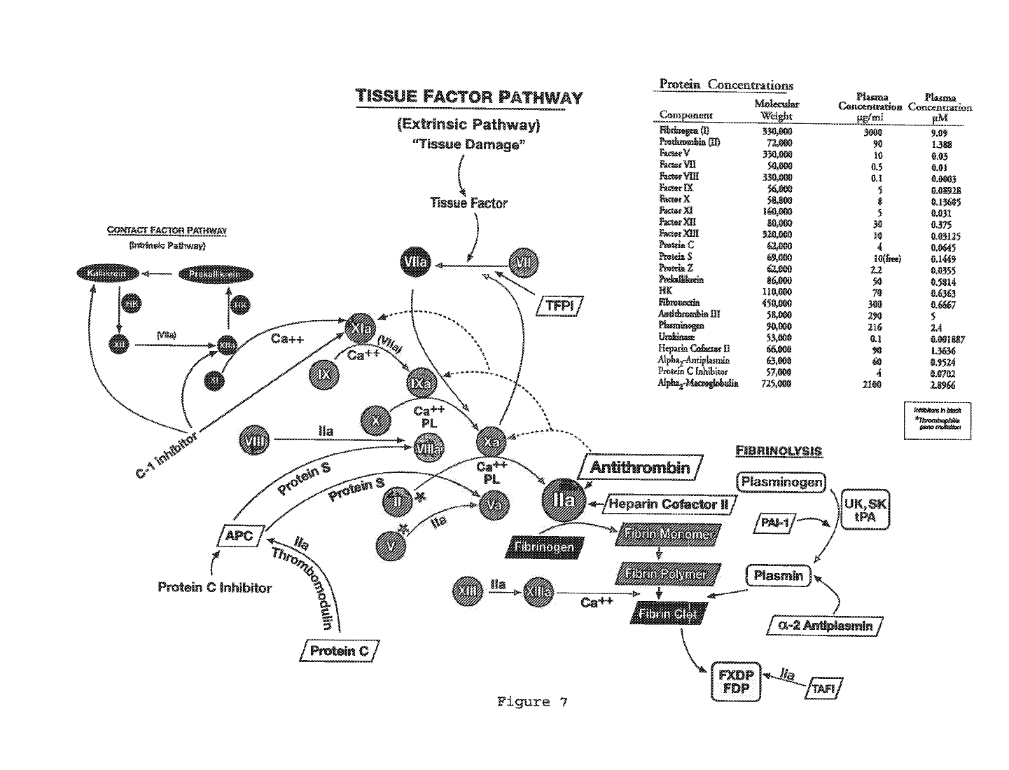

The methods and assays of the present invention are based on the principle that inhibition of serine protease (SP) activity by a serine protease inhibitor (SERPIN) under optimal concentrations of the serine protease and the SERPIN is accelerated by the presence of GAGs (e.g., DS and/or HS). The assay reaction is depicted below,

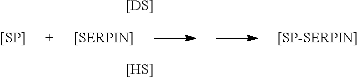

wherein DS is dermatan sulfate; HS is heparan sulfate; SP is active serine protease enzyme; SERPIN is serine protease inhibitor; and SP-SERPIN is the inactive serine protease/SERPIN-complex. It should be appreciated by those skilled in the art that any serine protease and any SERPIN involved in the proteolytic cascade of the coagulation pathway (e.g., blood clotting system) can be used in the assays and methods of the present invention. FIG. 7 illustrates the various enzymes SPs (e.g., Factor IXa, Xa, XIa and XIIa, etc.) and SERPINs (e.g., Antithrombin IIIa, Heparin Cofactor II, etc.) and enzymatic actions involved in the proteolytic cascade of the coagulation pathway which is described in further detail below.

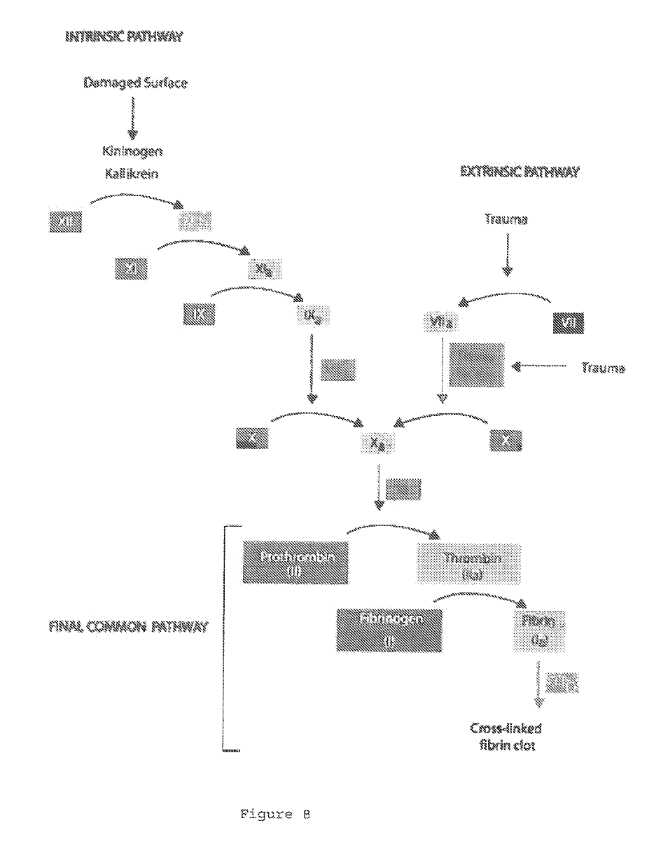

The blood clotting system or coagulation pathway, like the complement system, is a proteolytic cascade. Each enzyme of the pathway is present in the plasma as a zymogen (inactive form), which on activation undergoes proteolytic cleavage to release the active factor from the precursor molecule. The coagulation pathway functions as a series of positive and negative feedback loops which control the activation process. The ultimate goal of the pathway is to produce thrombin, which can then convert soluble fibrinogen into fibrin, which forms a clot. As illustrated in FIG. 8, the generation of thrombin can be divided into three phases, the intrinsic and extrinsic pathways, which provide alternative routes for the generation of factor X, and the final common pathway, which results in thrombin formation.

The intrinsic pathway is activated when blood comes into contact with sub-endothelial connective tissues or with negatively charged surfaces that are exposed as a result of tissue damage. Quantitatively it is the more important of the two pathways, but is slower to cleave fibrin than the extrinsic pathway. The Hageman factor (factor XII), factor XI, prekallikrein and high molecular weight kininogen (HMWK) are involved in this pathway of activation. The first step is the binding of factor XII to a sub-endothelial surface exposed by an injury. A complex of prekallikrein and HMWK also interacts with the exposed surface in close proximity to the bound factor XII, which becomes activated. During activation, the single chain protein of the native factor XII is cleaved into two chains (50 and 28 kDa), which remain linked by a disulphide bond. The light chain (28 kDa) contains the active site and the molecule is referred to as activated Hageman factor (factor XIIa). There is evidence that the Hageman factor can autoactivate, thus the pathway is self-amplifying.

Activated factor XII, in turn, activates prekallikrein. The kallikrein produced can then also cleave factor XII, and a further amplification mechanism is triggered. The activated factor XII remains in close contact with the activating surface, such that it can activate factor XI, the next step in the intrinsic pathway, which, to proceed efficiently, requires calcium. Also involved at this stage is HMWK, which binds to factor XI and facilitates the activation process. Activated factors XIa, XIIa and kallikrein are all serine proteases.

The intrinsic pathway ultimately activates factor X, a process which can also be brought about by the extrinsic pathway. Factor X is the first molecule of the common pathway and is activated by a complex of molecules containing activated factor IX, factor VIII, calcium and phospholipid, which is provided by the platelet surface, where this reaction usually takes place. The precise role of factor VIII in this reaction is not clearly understood. Its presence in the complex is essential, as evidenced by the consequences of factor VIII deficiency experienced by haemophiliacs. Factor VIII is modified by thrombin, a reaction that results in greatly enhanced factor VIII activity, promoting the activation of factor X.

The extrinsic pathway is an alternative route for the activation of the clotting cascade. It provides a very rapid response to tissue injury, generating activated factor X almost instantaneously, compared with the seconds, or even minutes, required for the intrinsic pathway to activate factor X. The main function of the extrinsic pathway is to augment the activity of the intrinsic pathway.

There are two components unique to the extrinsic pathway, tissue factor or factor III, and factor VII. Tissue factor is present in most human cells bound to the cell membrane. Once activated, tissue factor binds rapidly to factor VII, which is then activated to form a complex of tissue factor, activated factor VII, calcium and a phospholipid, and this complex then rapidly activates factor X.

As shown in FIG. 8, the intrinsic and extrinsic systems converge at factor X to a single common pathway which is responsible for the production of thrombin (factor IIa). In certain embodiments, the methods and assays of the present invention are based on the principle that the inactivation of thrombin (T) by heparin cofactor-II (HC) under optimal concentrations of T and HC is accelerated by the presence of, e.g., DS and/or HS. Under these assay conditions, the concentration and/or quantity of DS and/or HS is the reaction rate limiting factor in the inhibition of T. The assay reaction is depicted below:

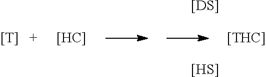

wherein DS is dermatan sulfate; HS is heparan sulfate; T is active thrombin enzyme; HC is thrombin inhibitor heparin cofactor II; and THC is the inactive thrombin/heparin cofactor II-complex.

Heparin cofactor-II (HC) belongs to a family of serine protease inhibitors (SERPINS) and its primary function is as an inhibitor of the enzyme thrombin (T). As used herein, the term “SERPIN” refers to serine protease inhibitors which are exemplified by species such as Heparin cofactor-II (HC). Heparin cofactor-II acts as an inhibitor of thrombin, and the inhibition of thrombin by heparin cofactor-II is accelerated by the presence of the GAGs DS and/or HS under conditions described herein.

Thrombin is a coagulation protein that affects the coagulation cascade and blood clotting. Thrombin is a serine protease and is responsible for converting soluble fibrinogen into insoluble strands of fibrin, as well as catalyzing many other coagulation-related reactions. The inhibition of thrombin by a SERPIN inhibitor results in an anticoagulant effect and this reaction is catalyzed by the GAGs DS, and to a lesser extent by HS. Thrombin is capable of cleaving thrombin substrates (e.g., a chromogenic thrombin substrate such as thrombin substrate S2238 which produces a chromogen with a measurable optical density at 405 nm). In accordance with the present invention, the inactivation of thrombin by heparin cofactor-II in the presence of DS and/or HS reduces the cleavage of the thrombin substrate. Accordingly, the methods and assays of the present invention provide means of quantifying the consumption of the chromogenic thrombin substrate (i.e., correlating to the inactivation of thrombin), for example using spectroscopy, and thus providing an indirect measurement of the concentration and/or quantity of DS and/or HS in the biological sample.

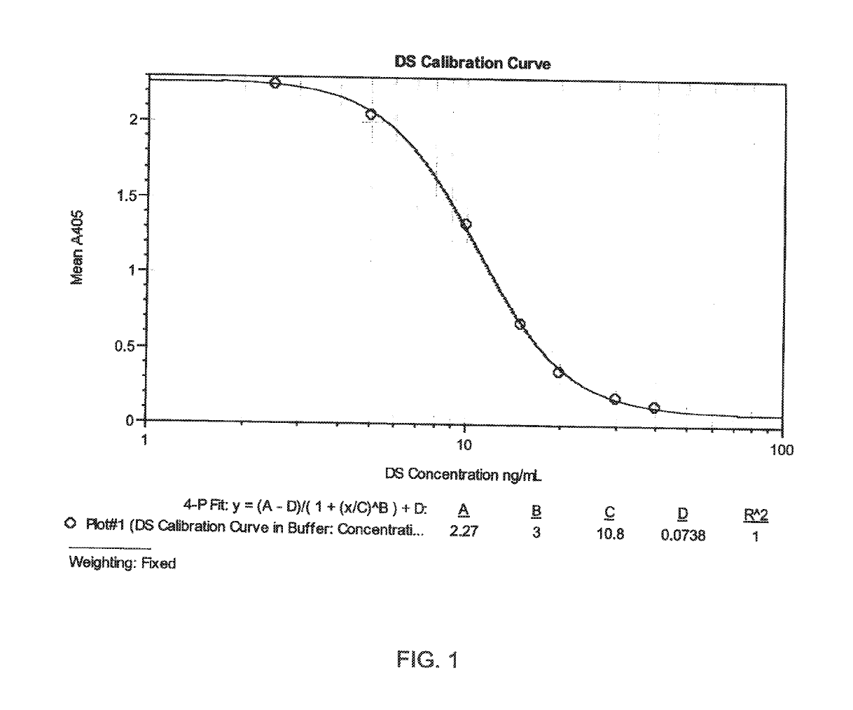

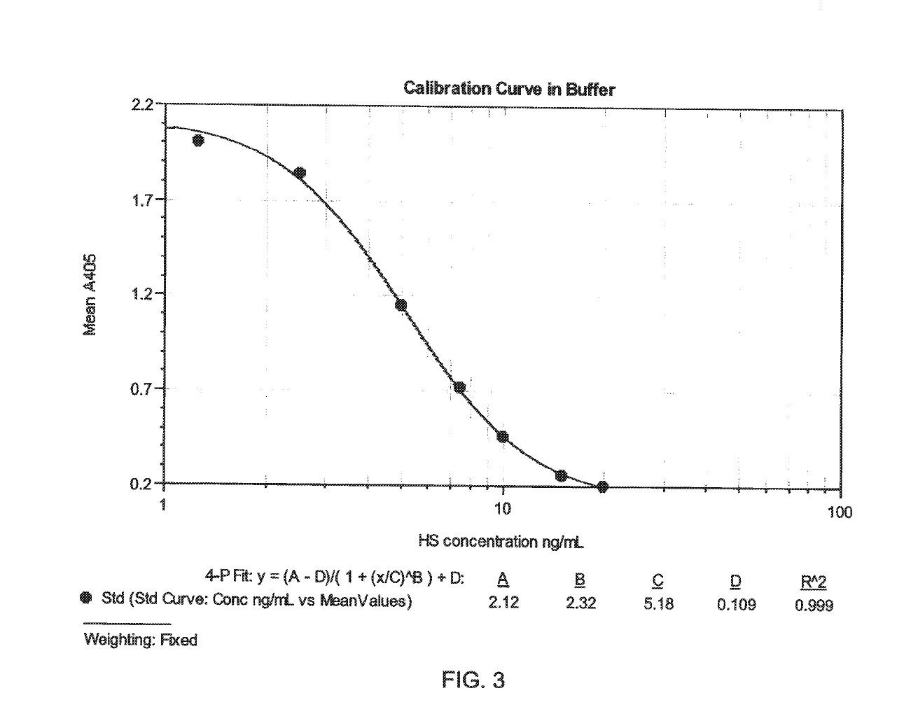

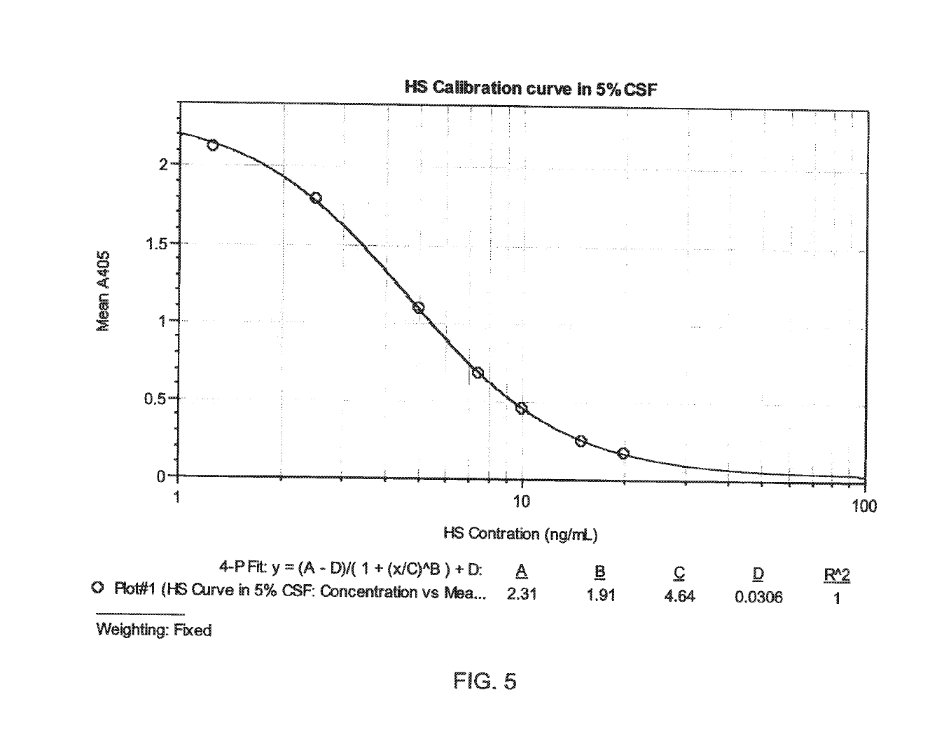

In accordance with the present invention, as the DS/HS concentration increases in the biological sample, inhibition of thrombin increases and less thrombin substrate is cleaved by thrombin. The reduced cleavage of thrombin substrate by thrombin as a result of increasing DS/HS concentrations present in the biological sample is illustrated by decreasing absorbance measurements obtained by spectrophotometric detection at 405 nm, as shown in FIG. 1, FIG. 3, and FIG. 5. The absorbance of the chromogenic thrombin substrate may be determined and compared to a standard or calibration curve prepared by plotting the mean absorbance value for known standard solutions versus the corresponding concentrations of such standard solutions. As the phrase is used herein, a “calibration curve” refers to a graphical plot of two or more variables (e.g., known concentration of a GAG and optical density of a chromogen). Provided in FIGS. 1, 3 and 5 are calibration curves prepared using HS and DS standard solutions in accordance with the examples provided herein, providing the ability to comparatively assess the absorbance values of a biological sample and thereby quantify the DS and HS concentrations in that biological sample (e.g., urine or CSF). In one embodiment of the present invention, the calibration curves are preferably prepared using standards with known concentrations of one or more GAGs, such that the optical density of a sample may be readily correlated with the calibration curve and the concentration or quantity of GAG in such sample may be readily determined. Quantitative assessments of GAGs in a biological sample thereby provide a means of assessing MPS disease progression or treatment efficacy.

In certain embodiments, the concentrations of glycosaminoglycan (e.g., heparan sulfate or dermatan sulfate) in one or more biological samples may be determined with reference to one or more calibration curves that are prepared to correlate spectrophotometric absorbance of a sample with known glycosaminoglycan concentrations. For example, calibration curves may be prepared using HS calibrator samples that are serially diluted from about 20 ng/mL to about 0.6 ng/mL in a selected assay buffer solution. Generally, the serially diluted calibrator samples may then be incubated with heparin cofactor II (e.g., at a final concentration of 4.68 μg/mL), followed by the addition of human thrombin (e.g., at a final concentration of about 0.1-0.2 μg/mL, such as about 0.15 μg/mL), followed by the addition of a suitable thrombin substrate and the absorbance determined on a plate reader.

In some embodiments, the assays and methods of the present invention contemplate comparative analyses of the sample relative to one or more controls. For example, comparing a biological sample relative to a positive control (e.g., a biological sample obtained from a subject with MPS) may be indicative of the presence of MPS. Alternatively, a comparison of a biological sample with a negative control (e.g., a biological sample obtained from a subject without MPS) may be indicative of the absence of MPS. In another embodiment, the positive and negative controls may be used to construct a calibration or standards curve to which the sample may be compared to assess the presence or absence of MPS.

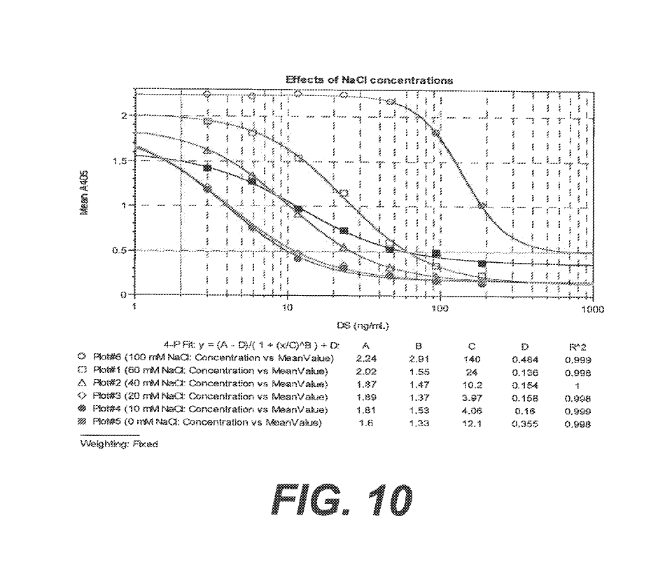

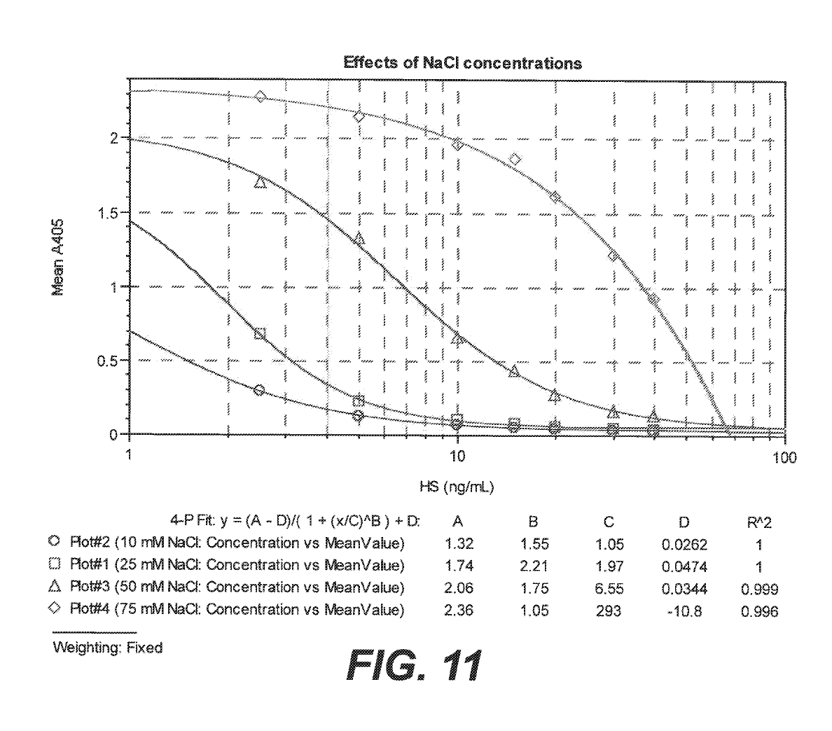

In certain embodiments, the assay buffer solutions (e.g., an assay buffer solution comprising HEPES, NaCl and PEG) used in the thrombin-coupled assays of the present invention provide a means of optimizing the sensitivity of the assays disclosed herein. The present inventors have discovered that the concentration of sodium chloride (NaCl) in the assay buffer solution affects the sensitivity of the thrombin-coupled assays. Accordingly, the properties of the assay buffer solution, and in particular the concentration of sodium chloride in such assay buffer solution, provides a means of controlling, modulating or otherwise improving the sensitivity of the thrombin-coupled assays. For example, as illustrated in FIG. 10, in certain instances the sensitivity of the thrombin-coupled assays to detect dermatan sulfate (DS) is a function of the concentration of sodium chloride in the assay buffer. In some embodiments, an assay buffer comprising between about 35-70 mM sodium chloride (e.g., about 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM, 60 mM, 61 mM, 62 mM, 63 mM, 64 mM, 65 mM, 66 mM, 67 mM, 68 mM or 69 mM of NaCl) provides optimum sensitivity facilitating the detection of dermatan sulfate in a sample (e.g., plasma or cerebrospinal fluid). Similarly, as illustrated in FIG. 11, in certain instances the sensitivity of the thrombin-coupled assays to detect heparan sulfate (HS) is a function of the concentration of sodium chloride in the assay buffer. In some embodiments, an assay buffer comprising between about 40-60 mM sodium chloride (e.g., about 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM or 60 mM of NaCl) provides optimum sensitivity facilitating the detection of heparan sulfate in a sample. In some embodiments, an assay buffer comprising between about 30-60 mM sodium chloride (e.g., about 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM or 60 mM of NaCl) provides optimum sensitivity, facilitating the detection of heparan sulfate in a sample.

In some embodiments, the assay buffer solution comprises HEPES, PEG (e.g., PEG-8000) and NaCI (e.g., about 40-60 mM NaCl). For example, an assay buffer solution comprising 10 mM HEPES, 50 mM NaCl and 0.25 mg/mL PEG-8000 at pH 7.5±0.05. Alternatively, another assay buffer solution may comprise 10 mM HEPES, 40 mM NaCl and 0.25 mg/mL PEG-8000 at pH 7.5±0.05. While certain methods, assays and kits of the present invention have been described with specificity in accordance with certain embodiments, the following examples serve only to illustrate the compounds of the invention and are not intended to limit the same.

The assay buffer solution (e.g., an assay buffer solution comprising NaCl, HEPES and PEG-8000) may be used to modulate the sensitivity of the assays disclosed herein, and in particular to modulate the ability of such assays to detect the concentration of one or more glycosaminoglycans in a biological sample (e.g., CSF). In certain embodiments, the assays disclosed herein are capable of determining the concentration of one or more glycosaminoglycans (e.g., heparan sulfate) present at less than about 3 ng/mL in a sample. Similarly, in other embodiments the assays disclosed herein are capable of determining the concentration of the one or more glycosaminoglycans (e.g., dermatan sulfate) present at less than about 2 ng/mL in a sample. In yet another embodiment, the assays disclosed herein are capable of determining the concentration of one or more glycosaminoglycans present at less than about 1 ng/mL in a sample. In still another embodiments, the assays disclosed herein are capable of determining the concentration of one or more glycosaminoglycans present at at least 0.5 ng/mL in a sample (e.g., at at least about 0.6 ng/mL or 0.75 ng/mL in the sample).

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or the entire group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. The publications, websites and other reference materials referenced herein to describe the background of the invention and to provide additional detail regarding its practice are hereby incorporated by reference.

### EXAMPLES

### Assay Materials and Methods

The assays described in the following examples were conducted to assess the concentrations of total glycosaminoglycans and DS or HS in biological samples (e.g., urine and cerebrospinal fluid (CSF)) by using an exemplary serine protease-based assay as described herein. Spectrophotometric detection was performed using Molecular Devices SPECTRAmax plate reader with attached computer equipped with SOFTMax Pro software and assay template, however, any similar device suitable for performing spectrophotometric detection can be used in the methods, assays and kits of the present invention. Chemicals and Reagents used in the assays included: Thrombin Human Alpha (commercially available from Enzyme Research Catalog #HT 1002a); Human Heparin Cofactor 2 (commercially available from Haematologic Technologies Inc, Catalog #HCII-0190); Thrombin Substrate S-2238 (commercially available from Chromogenix-Diapharma Catalog #82-0324-39) reconstituted in purified water; Dermatan Sulfate from pig mucosa (commercially available from Iduron, Catalog #GAG-DS01); Heparan Sulfate sodium salt, from bovine kidney, (commercially available from Sigma, Catalog #H7640-1MG, CAS: 57459-72-0); Recombinant Chondroitinase B (commercially available from Ibex Catalog #50-018, CAS:52227-83-5) diluted in Assay Buffer 1; Recombinant Heparinase I, II, III mixture (commercially available from Ibex, Catalog #50-010, CAS:9025-39-2 for Heparinase I, #50-011, CAS:na for Heparinase II and #50-012, CAS:37290-86-1 for Heparinase III) diluted in Assay Buffer 2; Polythylene Glycol 8000 (PEG-8000) (commercially available from Fisher Catalog #BP233-100, CAS: 25322-68-3); HEPES (commercially available from Acros, Catalog #172571000, CAS:7365-45-9); Sodium Chloride (NaCl) (commercially available from Fisher catalog #S640-500, CAS:7647-14-5); Glacial Acetic Acid (commercially available from Fisher Catalog #A38-500 CAS:64-19-7); Purified water: Milli-Q or better grade; Assay Buffer 1 solution comprised of 10 mM HEPES, 50 mM NaCl and 0.25 mg/mL PEG-8000 at pH 7.5±0.05; Assay Buffer 2 solution comprised of 10 mM HEPES, 40 mM NaCl and 0.25 mg/mL PEG-8000 at pH 7.5±0.05; and a Stop Buffer comprised of 20% Acetic Acid in water.

The preparation of Positive and Negative Controls was as follows. The Positive DS Control Urine was obtained from a subject with MPS II and diluted in Assay Buffer 1. The Positive DS Control CSF was obtained from a subject with MPS II and diluted in Assay Buffer 1. The Positive HS Control Urine was obtained from a subject with MPS IIIA and diluted in Assay Buffer 2. The Positive HS Control CSF was obtained from a subject with MPS IIIA and diluted in Assay Buffer 2. The Negative DS Control Urine was obtained from a normal subject and diluted in Assay Buffer 1. The Negative DS Control CSF was obtained from a normal subject and diluted in Assay Buffer 1. The Negative HS Control Urine was obtained from a normal subject and diluted in Assay Buffer 2. The Negative HS Control CSF was obtained from a normal subject and diluted in Assay Buffer 2. It should be noted that in lieu of obtaining a positive biological sample such as urine or CSF, the DS and HS positive controls can also be prepared by diluting purified DS and HS, respectively in an appropriate media.

### Example 1

The purpose of the following assay was to determine the concentration in a urine sample of total GAGs and dermatan sulfate (DS) by a thrombin-coupled method.

Urine unknown samples were diluted 1:100 in Assay Buffer 1 and treated at 37° C. for 2 hours with chondroitinase B (at a final concentration of 13 ng/mL) or left untreated, and incubated at 37° C. for 2 hours. It should be appreciated by those of ordinary skill in the art that dilution of the samples is not limited to 1:100, as such dilution is provided as an illustrative example. Following this incubation step, unknown urine samples, positive and negative DS urine controls and DS calibrator samples (serially diluted from 40 ng/mL to 2.5 ng/mL, i.e. 1 nM to 0.063 nM, in Assay Buffer 1) were incubated with heparin cofactor II (at a final concentration of 4.68 μg/mL i.e. 71.4 nM) at 37° C. for 5 minutes. Human thrombin was added to all tubes at a final concentration of 0.15 μg/mL i.e. 4 nM and all tubes were incubated at 37° C. for an additional 15 minutes. Thrombin substrate was then added to all tubes at a final concentration of 0.5 mM and incubated at 37° C. for 30 minutes. The reaction was stopped by adding Stop Buffer solution. The signal was then read on a plate reader at a wavelength of 405 nm.

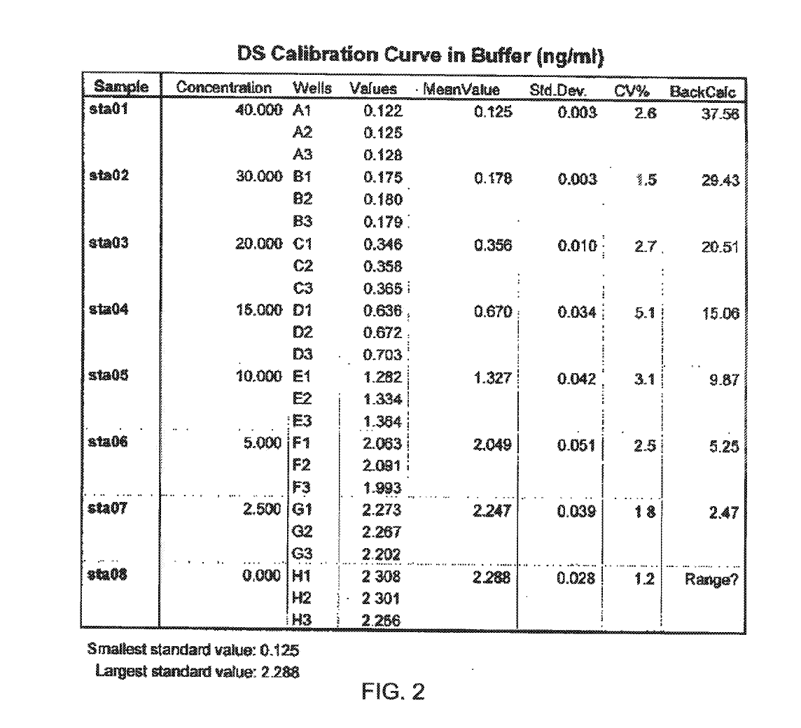

The absorbance value for each sample was then correlated to the dermatan sulfate calibrator curve (as in the example shown in FIG. 1) and the results reported as ng/mL of GAGs in each sample (as in the example shown in FIG. 2). For each unknown sample, the concentration of total GAGs is the value obtained from the sample left untreated by chondroitinase B (Table 2). The concentration of DS is identified using the value obtained from subtracting the value obtained from the sample treated by chondroitinase B from the total GAGs value (Table 3), followed by the multiplication of such value by an assay dilution factor of 2.67 (Table 4).

### Example 2

The purpose of the following assay was to determine the concentration in a cerebrospinal fluid (CSF) sample of total Glycosaminoglycans (GAGs) and dermatan sulfate (DS) by a thrombin-coupled method.

CSF unknown samples were diluted 1:5 in Assay Buffer 1 and treated at 37° C. for 2 hours with chondroitinase B (at a final concentration of 13 ng/mL) or left untreated, and incubated at 37° C. for 2 hours. It should be appreciated by those of ordinary skill in the art that dilution of the samples is not limited to 1:5, as such dilution is provided as an illustrative example. Following this incubation step, unknown CSF samples, positive and negative DS CSF controls and DS calibrator samples (serially diluted from 40 ng/mL to 2.5 ng/mL, i.e. 1 nM to 0.063 nM, in Assay Buffer 1) were incubated with heparin cofactor II (at a final concentration of 4.68 μg/mL i.e. 71.4 nM) at 37° C. for 5 minutes. Human thrombin was added to all tubes at a final concentration of 0.15 μg/mL (i.e. 4 nM) and all tubes were incubated at 37° C. for an additional 15 minutes. Thrombin substrate was then added to all tubes at a final concentration of 0.5 mM and incubated at 37° C. for 30 minutes. The reaction was stopped by adding Stop Buffer solution. The signal was then read on a plate reader at a wavelength of 405 nm.

The absorbance value for each sample was then correlated to the dermatan sulfate calibrator curve (as in the example shown in FIG. 1) and the results reported as ng/mL of GAGs in each sample (as in the example shown in FIG. 2). For each unknown sample, the concentration of total GAGs is the value obtained from the sample left untreated by chondroitinase B (Table 2). The concentration of DS is identified using the value obtained from subtracting the value obtained from the sample treated by chondroitinase B from the total GAGs value (Table 3), followed by the multiplication of such value by an assay dilution factor of 2.67 (Table 4).

TABLE 2
Sample absorbance values for total GAGs and GAGs after
Chondroitinase B treatment

| | | | Absorbance at 405 nm | |
|---|---|---|---|---|
| Sample Type | Sample No. | Dilution | Total GAG | Chondroitinase B |
| CSF | A | 10 | 0.720 | 2.078 |
| CSF | B | 15 | 0.333 | 2.197 |
| CSF | C | 20 | 0.490 | 2.197 |
| CSF | D | 20 | 0.495 | 2.198 |
| Urine | E | 400 | 0.425 | 2.024 |
| Urine | F | 200 | 0.441 | 2.137 |
| Urine | G | 800 | 0.292 | 2.233 |

TABLE 3
Sample GAG concentration values for total GAGs, GAGs after
Chondroitinase B treatment, and final DS values.

| Sample | Sample | | Concentration (ng/mL) | | |
|---|---|---|---|---|---|
| Type | No. | Dilution | Total GAG | Chondroitinase B | DS |
| CSF | A | 10 | 259.10 | 49.30 | 209.80 |
| CSF | B | 15 | 316.85 | ND | 316.85 |
| CSF | C | 20 | 332.40 | ND | 332.40 |
| CSF | D | 20 | 331.00 | ND | 331 |
| Urine | E | 400 | 7144 | 1640 | 5504 |
| Urine | F | 200 | 3704 | 876 | 2828 |
| Urine | G | 800 | 18072 | 2280 | 15792 |

ND: Not Detectable

TABLE 4
Sample GAG concentration values for total GAGs, GAGs after
Chondroitinase B treatment, and final DS values multiplied by an assay
dilution factor of 2.67.

| Sample | Sample | | Concentration (ng/mL) in sample | | |
|---|---|---|---|---|---|
| Type | No. | Dilution | Total GAG | Chondroitinase B | DS |
| CSF | A | 10 | 691.8 | 131.6 | 560.2 |
| CSF | B | 15 | 855.5 | ND | 855.5 |
| CSF | C | 20 | 892.6 | ND | 892.6 |
| CSF | D | 20 | 888.6 | ND | 888.6 |
| Urine | E | 400 | 19201.8 | 4451 | 14750.9 |
| Urine | F | 200 | 9888.1 | 2339 | 7549.2 |
| Urine | G | 800 | 48258.6 | 6093 | 42165.7 |

ND: Not Detectable

### Example 3

The purpose of the following assay was to determine the concentration in a urine sample of total GAGs and heparan sulfate (HS) by a thrombin-coupled method.

Urine unknown samples were diluted 1:100 in Assay Buffer 2 and treated at 37° C. for 2 hours with heparinase I, II, and III mixture (at a final concentration of 180 ng/mL) or left untreated, and incubated at 37° C. for 2 hours. It should be appreciated by those of ordinary skill in the art that dilution of the samples is not limited to 1:100, as such dilution is provided as an illustrative example. Following this incubation step, unknown urine samples, positive and negative HS urine controls and HS calibrator samples (serially diluted from 20 ng/mL to 1.25 ng/mL in Assay Buffer 2) were incubated with heparin cofactor II (at a final concentration of 4.68 μg/mL i.e. 71.4 nM) at 37° C. for 5 minutes. Human thrombin was added to all tubes at a final concentration of 0.15 μg/mL i.e. 4 nM and all tubes were incubated at 37° C. for an additional 15 minutes. Thrombin substrate was then added to all tubes at a final concentration of 0.5 mM and incubated at 37° C. for 30 minutes. The reaction was stopped by adding Stop Buffer solution. The signal was then read on a plate reader at a wavelength of 405 nm.

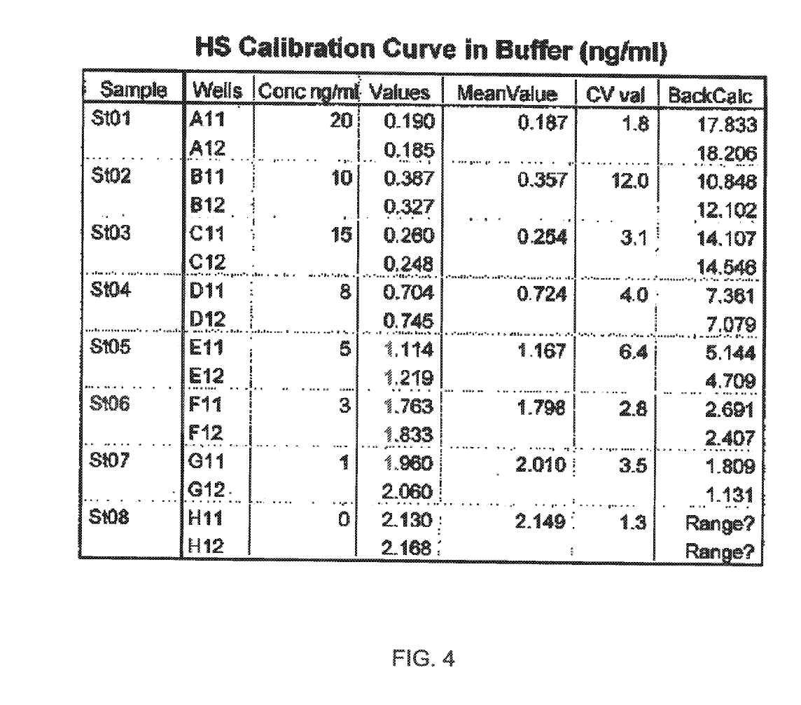

The absorbance value for each sample was then correlated to the heparan sulfate calibrator curve (as in the example shown in FIG. 3) and the results reported as ng/mL of GAGs in each sample (as in the example shown in FIG. 4). For each unknown sample, the concentration of total GAGs is the value obtained from the sample left untreated by the heparinase mixture (Table 5). The concentration of HS is identified using the value obtained from subtracting the value obtained from the sample treated by the heparinase mixture from the total GAGs value (Table 6), followed by the multiplication of such value by an assay dilution factor of 2.67 (Table 7).

### Example 4

The purpose of the following assay was to determine the concentration of total GAGs and heparan sulfate (HS) in a CSF sample by a thrombin-coupled method.

CSF samples were diluted 1:20 in Assay Buffer 2 and treated at 37° C. for 2 hours with a heparinase I, II, and III mixture (at a final concentration of 180 ng/mL) or left untreated and incubated at 37° C. for 2 hours. It should be appreciated by those of ordinary skill in the art that dilution of the samples is not limited to 1:20, as such dilution is provided as an illustrative example. Following this incubation step, unknown CSF samples, positive and negative HS CSF controls and HS calibrator samples (serially diluted from 20 ng/mL to 1.25 ng/mL in 1:20 normal pooled human CSF in Assay Buffer 2) were incubated with heparin cofactor II (at a final concentration of 4.68 μg/mL i.e. 71.4 nM) at 37° C. for 5 minutes. Human thrombin was added to all tubes at a final concentration of 0.15 μg/mL (i.e. 4 nM) and all tubes were incubated at 37° C. for an additional 15 minutes. Thrombin substrate was then added to all tubes at a final concentration of 0.5 mM and incubated at 37° C. for 30 minutes. The reaction was stopped by adding Stop Buffer solution. The signal was then read on a plate reader at a wavelength of 405 nm.

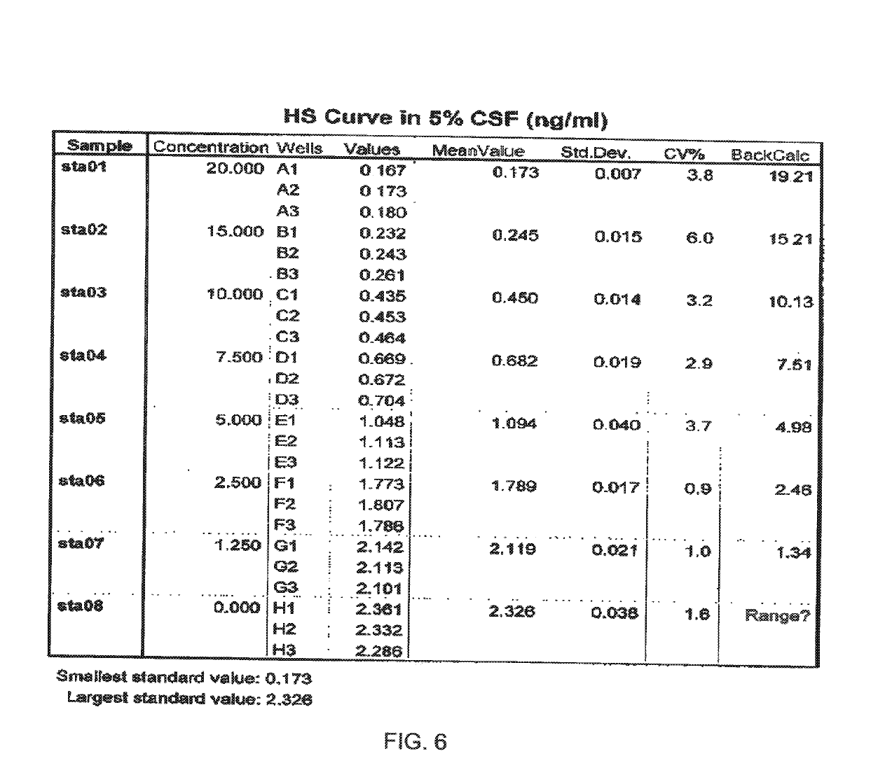

The absorbance value for each sample was then correlated to the heparan sulfate calibrator curve (as in the example shown in FIG. 5) and the results reported as ng/mL of GAGs in each sample (as shown in the example shown in FIG. 6). For each unknown sample, the concentration of total GAGs is the value obtained from the sample left untreated by the heparinase mixture (Table 5). The concentration of HS is identified using the value obtained from subtracting the value obtained from the sample treated by the heparinase mixture from the total GAGs value (Table 6), followed by the multiplication of such value by an assay dilution factor of 2.67 (Table 7).

TABLE 5
Sample GAG absorbance values for total GAGs, GAGs after Heparinase
mixture treatment.

| | | | Absorbance at 405 nm | |
|---|---|---|---|---|
| Sample Type | Sample No. | Dilution | Total GAG | Heparinase Mixture |
| CSF | H | 20 | 0.709 | 2.08 |
| CSF | I | 20 | 0.996 | 2.156 |
| CSF | J | 20 | 0.976 | 2.089 |
| CSF | K | 20 | 1.051 | 2.141 |
| Urine | L | 800 | 0.548 | 2.069 |
| Urine | M | 3200 | 0.462 | 2.072 |
| Urine | N | 3200 | 0.686 | 2.13 |

TABLE 6
Sample GAG concentration values for total GAGs, GAGs after Heparinase
mixture treatment, and final HS values.

| | | | Concentration (ng/mL) | | |
|---|---|---|---|---|---|
| Sample | Sample | | Total | | |
| Type | No. | Dilution | GAG | Heparinase Mixture | HS |
| CSF | H | 20 | 144.10 | ND | 144.10 |
| CSF | I | 20 | 173.80 | 35.0 | 138.80 |
| CSF | J | 20 | 132.20 | ND | 132.20 |
| CSF | K | 20 | 158.10 | 38.9 | 119.20 |
| Urine | L | 800 | 7466.32 | ND | 7466.32 |
| Urine | M | 3200 | 33949.82 | ND | 33949.82 |
| Urine | N | 3200 | 25334.60 | ND | 25334.60 |

ND: Not Detectable

TABLE 7
Sample GAG concentration values for total GAGs, GAGs after Heparinase
mixture treatment, and final HS values multiplied by an assay dilution
factor of 2.67.

| Sample | Sample | | Concentration (ng/mL) in sample | | |
|---|---|---|---|---|---|
| Type | No. | Dilution | Total GAG | Heparinase Mixture | HS |
| CSF | H | 20 | 433.4 | ND | 433.4 |
| CSF | I | 20 | 314.6 | ND | 314.6 |
| CSF | J | 20 | 300.3 | ND | 300.3 |
| CSF | K | 20 | 275.8 | ND | 275.8 |
| Urine | L | 800 | 19935.1 | ND | 19935.1 |
| Urine | M | 3200 | 90646.0 | ND | 90646.0 |
| Urine | N | 3200 | 67643.4 | ND | 67643.4 |

ND: Not Detectable

### Example 5

MPS-IIIA, which is also known as Sanfilippo syndrome A, is a rare autosomal recessive lysosomal storage disease, caused by a deficiency in one of the enzymes needed to break down the glycosaminoglycan (GAG) heparan sulfate (HS). MPS-IIIA has been shown to occur as a result of 70 different possible mutations in the heparan N-sulfatase gene, which reduce enzyme function and cause an accumulation of HS in affected patients. To better understand the underlying pathology of MPS-IIIA, the inventors have conducted the present observational natural history study of MPS-IIIA using a thrombin-coupled assay of the present invention. The objectives of the study were to define a series of objective clinical parameters that could be used to monitor disease progression over a 12 month period and to develop a better understanding of the MPS-IIIA clinical disease spectrum.

To conduct the present study, a total of twenty-five geographically diverse subjects with a confirmed diagnosis of MPS-IIIA were recruited. Each MPS-IIIA subject was required to have a calendar and developmental age, each greater than 1 year. The control group for the study was comprised of twenty young healthy adult subjects, from a wide geographical distribution from across North America. Subjects were evaluated upon enrollment and at six and twelve months, and were subjected to a comprehensive neurodevelopmental assessment and brain imaging. Upon evaluation, each subject underwent a lumbar puncture to obtain a cerebrospinal fluid (CSF) sample from the lumbar intrathecal space, and the sample was immediately frozen at −80° C. in 1.0 ml aliquots. CSF was selected as the biological sample because its constituents exist in a dynamic equilibrium with those in the central nervous system. The concentrations of GAGs in CSF were measured using the assay described in Example 4.

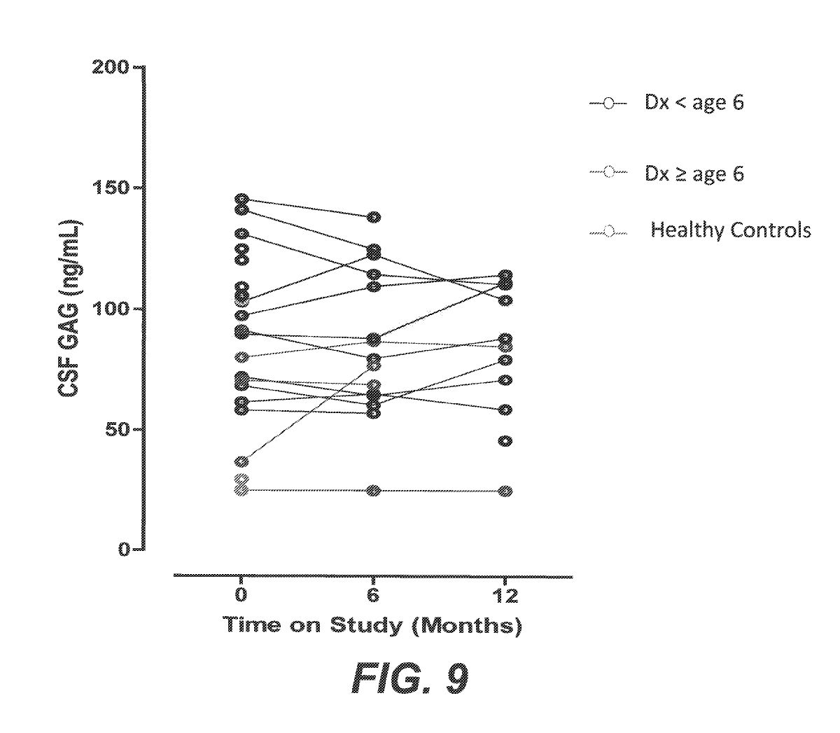

The results of the ongoing study segmented by: (i) MPS-IIIA subjects younger than 6 years old (Dx<6), (ii) MPS-IIIA subjects 6 years of age or older (Dx≧6) and (iii) healthy subjects, are presented in FIG. 9. As illustrated in FIG. 9, elevated concentrations of GAGs were observed in those MPS-IIIA subjects evaluated. Furthermore, the concentrations of GAGs in those MPS-IIIA subjects appear to remain at a relatively constant level for up to twelve months.

The foregoing therefore confirms the ability of the thrombin-coupled assays disclosed herein to measure GAG concentration in a biological sample and furthermore illustrates that GAG concentrations and in particular HS concentrations, in a biological sample (e.g., CSF) may be used as an objective indicator to characterize mucopolysaccharidosis severity and to monitor clinical responsiveness to therapeutic intervention.

### Example 6

The purpose of the present studies was to determine whether and to what extent the sodium chloride (NaCl) concentration of the assay buffer solution impacts the sensitivity of the present thrombin-coupled assays. To determine the effect of NaCl concentration in the assay buffer, six different dermatan sulfate (DS) calibration curves ranging from 3 ng/mL to 187 ng/mL were generated using assay buffer solutions having NaCl concentrations ranging from 0.0 mM to 100 mM.

The assays were conducted in accordance with the assay procedures generally described above using the DS reference standards and the signal was read on a microplate reader at a wavelength of 405 nm. The DS calibration curves were prepared by diluting the DS stock solution (2 mg/mL) to 20 μg/mL by mixing 10 μL of the DS stock solution with 990 μL of one of the selected assay buffer solutions, followed by vortexing gently to mix. The diluted DS stock solutions were subject further dilution to prepare diluted working solutions having final DS concentrations ranging from 3-187 ng/mL.

Working solutions of Heparin Cofactor II (HC-II) were then prepared by diluting a HC-II Stock Solution to 12.5 μg/mL using one of each the selected assay buffer solutions being evaluated. Next, 37.5 μL of the HC-II working solution was added in triplicate to each well of a microtiter plate accompanied by 37.5 μL of the selected diluted DS working solution. The microtiter plate was sealed and incubated at 37° C. for approximately 5 minutes with gentle shaking. After 5 minutes of incubation, 25 μL of a thrombin working solution (0.75 μg/mL) was quickly added to each well of the microtiter plate accompanied by 25 μL of one of each the selected assay buffer solutions. The microtiter plate was then re-sealed and incubated at 37° C. for approximately 15 minutes with gentle shaking. Following 15 minutes of incubation, 100 μL of a thrombin substrate (S-2238) working solution was quickly added to each well of the plate. The microtiter plate was then re-sealed and further incubated at approximately 37° C. for 30 minutes with gentle shaking. After 30 minutes of incubation, the reaction was stopped by adding 50 μL of the stop buffer solution (acetic acid) to each well of the microtiter plate. Within 10 minutes of stopping the reaction, the microtiter plate was read at a wavelength of 405 nm using a Molecular Devices SPECTRAMAX microplate reader using the SOFTMAX program.

As illustrated in FIG. 10, a lower percentage of thrombin inhibition was observed using a buffer solution that comprised 100 mM NaCl, whereas the buffer solution that excluded NaCl produced a relatively flat inhibition curve. In contrast, the buffer solutions having 40 mM and 60 mM NaCl produced sensitive and steep calibration curves. Based on these findings, it was determined that to achieve an optimum sensitivity of the thrombin-coupled assays to detect dermatan sulfate, the assay buffer solution should have about 40-60 mM NaCl. The optimized buffer solutions designated Assay Buffer 1 and Assay Buffer 2 (comprising 40 mM NaCl and 50 mM NaCl, respectively) demonstrated optimum sensitivity and were used to conduct the foregoing thrombin-coupled assays.

### Example 7

The purpose of the present studies was to determine whether and to what extent the sodium chloride (NaCl) concentration of the assay buffer solution impacts the sensitivity of the present thrombin-coupled assays. To determine the effect of NaCl concentration in the assay buffer, four different heparan sulfate (HS) thrombin inhibition calibration curves ranging from 2.5 ng/mL to 40 ng/mL were generated using assay buffer solutions having NaCl concentrations ranging from 10 mM to 75 mM.

The assays were conducted in accordance with the assay procedures generally described above using the HS reference standards and the signal was read on a microplate reader at a wavelength of 405 nm. The HS calibration curves were prepared by diluting the HS stock solution (1 mg/mL) to 20 μg/mL by mixing 10 μL of the HS stock solution with 490 μL of one of the selected assay buffer solutions, followed by vortexting gently to mix. The diluted HS stock solutions were subject to further dilution using assay buffer solutions to form working solutions having final HS concentrations ranging from 2.5-40 ng/mL.

Working solutions of Heparin Cofactor II (HC-II) were prepared by diluting a HC-II Stock Solution to 12.5 μg/mL using one of each the selected assay buffer solutions being evaluated. Next, 37.5 μL of the HC-II working solution was added in triplicate to each well of a microtiter plate accompanied by 37.5 μL of the selected diluted HS working solution. The microtiter plate was sealed and incubated at 37° C. for approximately 5 minutes with gentle shaking. After 5 minutes of incubation, 25 μL of a thrombin working solution (0.6 μg/mL) was quickly added to each well of the microtiter plate accompanied by 25 μL of one of each the selected assay buffer solutions. The microtiter plate was then re-sealed and incubated at 37° C. for approximately 15 minutes with gentle shaking. Following 15 minutes of incubation, 100 μL of a thrombin substrate (S-2238) working solution was quickly added to each well of the plate. The microtiter plate was then re-sealed and further incubated at approximately 37° C. for 30 minutes with gentle shaking. After 30 minutes of incubation, the reaction was stopped by adding 50 μL of the stop buffer solution (acetic acid) to each well of the microtiter plate. Within 10 minutes of stopping the reaction, the microtiter plate was read at a wavelength of 405 nm using a Molecular Devices SPECTRAMAX microplate reader using the SOFTMAX program.

As illustrated in FIG. 11, a lower percentage of thrombin inhibition was observed using a buffer solution that comprised 75 mM NaCl, whereas the buffer solutions that comprised 10 mM and 25 mM of NaCl produced relatively flat calibration curves. In contrast, the buffer solutions having 50 mM NaCl produced a sensitive and steep thrombin inhibition curve. Based on these findings, it was determined that to achieve an optimum sensitivity of the thrombin-coupled assays used to detect heparan sulfate, the buffer solution should have about 40-50 mM NaCl. The optimized buffer solutions designated Assay Buffer 2 (comprising 50 mM NaCl) demonstrated optimum sensitivity and was used to conduct the foregoing thrombin-coupled assays.

What is claimed is:

1. A method for determining the concentration of one or more glycosaminoglycans in a sample comprising: (a) combining a serine protease, a labeled substrate for the serine protease, an inhibitor of the serine protease, and a sample suspected of comprising one or more glycosaminoglycans in an assay buffer solution comprising NaCl, under conditions and for a time suitable for cleavage of the labeled substrate by the serine protease to produce a detectable signal; (b) detecting the detectable signal; and (c) comparing the amount of detectable signal with a standard to determine the concentration of the one or more glycosaminoglycans in the sample;
wherein the serine protease is a serine protease of the clotting cascade;
wherein the inhibitor of the serine protease is selected from the group consisting of heparin cofactor II and antithrombin III;
wherein the one or more glycosaminoglycans are selected from the group consisting of dermatan sulfate (DS) and heparan sulfate (HS); and
wherein the sensitivity of the method is modulated by the concentration of NaCl in the assay buffer solution.

2. A method according to claim 1 wherein the serine protease is selected from the group consisting of the serine proteases shown in FIG. 7 and FIG. 8.

3. A method according to claim 1 wherein the labeled substrate is a chromogenic or fluorogenic substrate.

4. A method according to claim 1 wherein the serine protease is thrombin and the labeled substrate is a chromogenic thrombin substrate.

5. A method according to claim 1 wherein the sample is treated to inactivate all but one of the glycosaminoglycans in the sample.

6. A method according to claim 5 wherein the sample is treated with chondroitinase B and the active glycosaminoglycan is dermatan sulfate.

7. A method according to claim 1 wherein detecting the detectable signal is performed by spectrophotometric detection.

8. A method according to claim 7 wherein the spectrophotometric detection is performed at 405 nm.

9. A method according to claim 1 wherein the sample is a biological sample.

10. A method according to claim 1 wherein the sample is selected from the group consisting of urine, serum, cerebrospinal fluid, and saliva.

11. A method according to claim 1 wherein the standard is a curve which calibrates spectrophotometric absorbance with glycosaminoglycan concentration.

12. A method according to claim 1, wherein the serine protease, labeled substrate for the serine protease and inhibitor of the serine protease are combined in the assay buffer solution prior to being combined with the sample suspected of comprising one or more glycosaminoglycans.

13. A method according to claim 12, wherein the assay buffer solution further comprises HEPES and PEG-8000.

14. A method according to claim 13, wherein the assay buffer solution comprises 40-60 mM NaCl.

15. A method according to claim 14, wherein the sample suspected of comprising one or more glycosaminoglycans is combined with the assay buffer solution prior to being combined with the serine protease, labeled substrate for the serine protease and inhibitor of the serine protease.

16. A method of identifying an individual having a mucopolysaccharidosis (MPS), comprising determining the concentration of one or more glycosaminoglycans in a biological sample obtained from the individual by a method comprising: (a) combining a serine protease, a labeled substrate for the serine protease, an inhibitor of the serine protease, and a biological sample from the subject in an assay buffer solution comprising NaCl, under conditions and for a time suitable for cleavage of the labeled substrate by the serine protease to produce a detectable signal; (b) detecting the detectable signal; and (c) comparing the amount of detectable signal with a standard to determine the concentration of the one or more glycosaminoglycans in the sample;
wherein the serine protease is a serine protease of the clotting cascade;
wherein the inhibitor of the serine protease is selected from the group consisting of heparin cofactor II and antithrombin III;
wherein the concentration of the one or more glycosaminoglycans is indicative of whether the individual has MPS; and
wherein the sensitivity of the method is a function of the concentration of NaCl in the assay buffer solution.

17. A method according to claim 16 wherein the serine protease is selected from the group consisting of the serine proteases shown in FIG. 7 and FIG. 8.

18. A method according to claim 16 wherein the labeled substrate is a chromogenic or fluorogenic substrate.

19. A method according to claim 16 wherein the serine protease is thrombin and the labeled substrate is a chromogenic thrombin substrate.

20. A method according to claim 16 wherein the sample is treated to inactivate all but one of the glycosaminoglycans in the sample.

21. A method according to claim 20 wherein the sample is treated with chondroitinase B and the active glycosaminoglycan is dermatan sulfate.

22. A method according to claim 16 wherein detecting the detectable signal is performed by spectrophotometric detection.

23. A method according to claim 22 wherein the spectrophotometric detection is performed at 405 nm.

24. A method according to claim 16 wherein the biological sample is selected from the group consisting of urine, serum, cerebrospinal fluid, and saliva.

25. A method according to claim 16 wherein the standard is a curve which calibrates spectrophotometric absorbance with glycosaminoglycan concentration.

26. A method according to claim 16, wherein the MPS is selected from the group consisting of MPS I, MPS II, MPS IIIA, MPS IIIB, MPS IIIC, MPS IIID, MPS IVA, MPS IVB, MPS VI, MPS VII and MPS IX.

27. A method according to claim 16, wherein the serine protease, labeled substrate for the serine protease and inhibitor of the serine protease are combined in the assay buffer solution prior to being combined with the biological sample from the subject.

28. A method according to claim 27, wherein the assay buffer solution further comprises HEPES and PEG-8000.

29. A method according to claim 28, wherein the assay buffer solution comprises 40-60 mM NaCl.

30. A method according to claim 28, wherein the assay buffer solution comprises about 50 mM NaCl.

31. A method according to claim 16, wherein the one or more glycosaminoglycans are selected from the group consisting of dermatan sulfate (DS), heparan sulfate (HS), chondroitin sulfate (CS), keratan sulfate (KS) and hyaluronan.

32. A method of determining the efficacy of one or more therapeutic agents or regimens for treatment of mucopolysaccharidosis (MPS) comprising determining the concentration of one or more glycosaminoglycans in a first biological sample obtained from an individual prior to administration of one or more therapeutic agents or regimens to the individual, determining the concentration of the glycosaminoglycans in a second biological sample obtained from the individual after administration of one or more therapeutic agents or regimens to the individual;
wherein if the concentration of the glycosaminoglycans in the second biological sample is lower than the concentration in the first biological sample the one or more therapeutic agents or regimens are efficacious for treatment of MPS; and
wherein determining the concentration of one or more glycosaminoglycans in the first and second biological samples is performed by a method comprising: (a) combining a serine protease, a labeled substrate for the serine protease, an inhibitor of the serine protease, and the first or second biological sample in an assay buffer solution comprising NaCl, under conditions and for a time suitable for cleavage of the labeled substrate by the serine protease to produce a detectable signal; (b) detecting the detectable signal; and (c) comparing the amount of detectable signal with a standard to determine the concentration of the one or more glycosaminoglycans in the sample;
wherein the serine protease is a serine protease of the clotting cascade;
wherein the inhibitor of the serine protease is selected from the group consisting of heparin cofactor II and antithrombin III;
wherein the one or more glycosaminoglycans are selected from the group consisting of dermatan sulfate (DS) and heparan sulfate (HS); and
wherein the sensitivity of the method is modulated by the concentration of NaCl in the assay buffer solution.

33. The method of claim 32, wherein the one or more therapeutic agents or regimens are selected from the group consisting of enzyme replacement therapies, bone marrow transplantation, and combinations thereof.

34. The method of claim 32, wherein the one or more therapeutic agents are selected from the group consisting of iduronate sulfatase, idursulfase, alpha-L-iduronidase, heparin sulfamidase, N-acetylglucosaminidase, N-acetylglucosamine 6-sulfatase, N-acetylgalactosamine-4-sulfatase and beta-glucoronidase.

35. A method according to claim 32 wherein the serine protease is selected from the group consisting of the serine proteases shown in FIG. 7 and FIG. 8.

36. A method according to claim 32 wherein the labeled substrate is a chromogenic or fluorogenic substrate.

37. A method according to claim 32 wherein the serine protease is thrombin and the labeled substrate is a chromogenic thrombin substrate.

38. A method according to claim 32 wherein detecting the detectable signal is performed by spectrophotometric detection.

39. A method according to claim 38 wherein the spectrophotometric detection is performed at 405 nm.

40. A method according to claim 32 wherein the first and second biological samples are selected from the group consisting of urine, serum, cerebrospinal fluid, and saliva.

41. A method according to claim 32 wherein the standard is a curve which calibrates spectrophotometric absorbance with glycosaminoglycan concentration.

42. A method according to claim 32, wherein the MPS is selected from the group consisting of MPS I, MPS II, MPS IIIA, MPS IIIB, MPS IIIC, MPS IIID, MPS IVA, MPS IVB, MPS VI, MPS VII and MPS IX.

43. A method of determining the progression of a mucopolysaccharidosis (MPS) disorder in an individual, comprising determining the concentration of one or more glycosaminoglycans in a first biological sample obtained from an individual and determining the concentration of the one or more glycosaminoglycans in one or more subsequent biological samples obtained from the individual;
wherein if the concentration of the one or more glycosaminoglycans in the one or more subsequent biological samples is greater than the concentration in the first biological sample it is indicative that the MPS is progressing; and
wherein determining the concentration of one or more glycosaminoglycans in the first and subsequent biological samples is performed by a method comprising (a) combining a serine protease, a labeled substrate for the serine protease, an inhibitor of the serine protease, and the first or subsequent biological sample in a buffer solution comprising NaCl, under conditions and for a time suitable for cleavage of the labeled substrate by the serine protease to produce a detectable signal; (b) detecting the detectable signal; and (c) comparing the amount of detectable signal with a standard to determine the concentration of the one or more glycosaminoglycans in the sample;
wherein the serine protease is a serine protease of the clotting cascade;
wherein the inhibitor of the serine protease is selected from the group consisting of heparin cofactor II and antithrombin III; and
wherein the sensitivity of the method is modulated by the concentration of NaCl in the buffer solution.

44. A method according to claim 43, wherein the one or more glycosaminoglycans are selected from the group consisting of dermatan sulfate (DS), heparan sulfate (HS), chondroitin sulfate (CS), keratan sulfate (KS) and hyaluronan.

45. A method according to claim 5 wherein the sample is treated with one or more heparinases and the active glycosaminoglycan is heparan sulfate.

46. A method according to claim 43, wherein the serine protease, labeled substrate for the serine protease and inhibitor of the serine protease are combined in an assay buffer solution.

47. A method according to claim 43, wherein the assay buffer solution further comprises HEPES and PEG-8000.

48. A method according to claim 47, wherein the assay buffer solution comprises 40-60 mM NaCl.

49. A method according to claim 47, wherein the sensitivity of the method to determine the concentration of the one or more glycosaminoglycans is a function of the concentration of NaCl in the assay buffer solution.

50. An assay for detecting the concentration of one or more glycosaminoglycans in a sample comprising: (a) combining a serine protease, a labeled substrate for the serine protease, an inhibitor of the serine protease, and a sample suspected of comprising one or more glycosaminoglycans in an assay buffer solution comprising NaCl, under conditions and for a time suitable for cleavage of the labeled substrate by the serine protease to produce a detectable signal; (b) detecting the detectable signal; and (c) comparing the amount of detectable signal with a standard to determine the concentration of the one or more glycosaminoglycans in the sample;
wherein the serine protease is a serine protease of the clotting cascade;
wherein the inhibitor of the serine protease is selected from the group consisting of heparin cofactor II and anti-thrombin III;
wherein the one or more glycosaminoglycans do not comprise heparin; and
wherein the sensitivity of the assay is modulated by the concentration of NaCl in the assay buffer solution.

51. An assay according to claim 50, wherein the assay is capable of determining the concentration of the one or more glycosaminoglycans present at less than about 3 ng/mL in the sample.

52. An assay according to claim 51, wherein the assay is capable of determining the concentration of the one or more glycosaminoglycans present at least about 0.6 ng/mL in the sample.

53. An assay according to claim 50, wherein the serine protease, the labeled substrate for the serine protease and the inhibitor of the serine protease are combined in an assay buffer solution.

54. An assay according to claim 53, wherein the assay buffer solution modulates the sensitivity of the assay to detect the one or more glycosaminoglycans in the sample.

55. An assay according to claim 54, wherein the assay buffer solution further comprises HEPES and PEG-8000.

56. An assay according to claim 55, wherein the concentration of NaCl is about 40 mM NaCl.

57. An assay according to claim 56, wherein the one or more glycosaminoglycans comprises dermatan sulfate (DS) and wherein the concentration of the NaCl in the assay buffer solution is about 40-60 mM.

58. An assay according to claim 56, wherein the one or more glycosaminoglycans comprises heparan sulfate (HS) and wherein the concentration of the NaCl in the assay buffer solution is about 45-55 mM.

59. An assay for determining the concentration of one or more glycosaminoglycans in a sample comprising: (a) combining a serine protease, a labeled substrate for the serine protease, an inhibitor of the serine protease, and a sample suspected of comprising one or more glycosaminoglycans in an assay buffer solution comprising about 40-60 mM NaCl under conditions and for a time suitable for cleavage of the labeled substrate by the serine protease to produce a detectable signal; (b) detecting the detectable signal; and (c) comparing the amount of detectable signal with a standard to determine the concentration of the one or more glycosaminoglycans in the sample;
wherein the serine protease is a serine protease of the clotting cascade;
wherein the inhibitor of the serine protease is selected from the group consisting of heparin cofactor II and anti-thrombin III; and
wherein the sensitivity of the assay is modulated by the concentration of NaCl in the assay buffer solution.

60. An assay according to claim 59, wherein the assay buffer solution modulates the sensitivity of the assay to determine the concentration of the one or more glycosaminoglycans.

61. An assay according to claim 59, wherein the buffer solution further comprises HEPES and PEG-8000.

62. An assay according to claim 59, wherein the assay is capable of determining the concentration of the one or more glycosaminoglycans present at less than about 3 ng/mL in the sample.

63. An assay according to claim 59, wherein the assay is capable of determining the concentration of the one or more glycosaminoglycans present at less than about 2 ng/mL in the sample.

64. An assay according to claim 59, wherein the assay is capable of determining the concentration of the one or more glycosaminoglycans present at less than about 1 ng/mL in the sample.

65. An assay according to claim 59, wherein the assay is capable of determining the concentration of the one or more glycosaminoglycans present at at least 0.5 ng/mL in the sample.

66. An assay according to claim 59, wherein the assay is capable of determining the concentration of the one or more glycosaminoglycans present at at least about 0.6 ng/mL in the sample.

67. An assay according to claim 59, wherein the assay is capable of determining the concentration of the one or more glycosaminoglycans present at at least about 0.75 ng/mL in the sample.

68. An assay according to claim 59, wherein the concentration of NaCl in the assay buffer solution modulates the sensitivity of the assay to determine the concentration one or more glycosaminoglycans in the sample.

69. An assay according to claim 59, wherein the one or more glycosaminoglycans comprise dermatan sulfate (DS) and wherein the assay buffer solution comprises about 50 mM of NaCl.

70. An assay according to claim 59, wherein the one or more glycosaminoglycans comprise heparan sulfate (HS) and wherein the assay buffer solution comprises about 45-55 mM of NaCl.
